(19) United States
(12) Patent Application Publication
(10) Pub. No.: US 2023/0274806 A1
(43) Pub. Date: Aug. 31, 2023

(54) DEVICE AND METHOD FOR PROVIDING PATIENT TRANSFER MEDIATION SERVICE

(71) Applicant: IKOOB CO., LTD, Seoul (KR)

(72) Inventors: Jae Hyoung CHO, Seoul (KR); Ji Hyeon JU, Seoul (KR)

(21) Appl. No.: 18/014,074

(22) PCT Filed: Jun. 30, 2020
(86) PCT No.: PCT/KR2020/008512

§ 371 Date: Dec. 30, 2022

(51) Int. Cl. G16H 10/60; G16H 15/00; G16H 40/20

(52) U.S. Cl. CPC G16H 10/60; G16H 15/00; G16H 40/20

(57) ABSTRACT

A device and a method for providing a patient transfer mediation service according to an embodiment of the present disclosure correspond to a patient transfer mediation service providing method of transferring a patient to a referral hospital by a terminal of a transfer-requesting hospital, wherein the method includes: outputting a provisionally-recommended referral hospital list including at least one provisionally-recommended referral hospital to which the patient is to be transferred; determining, from the output provisionally-recommended referral hospital list, a recommended referral hospital to which the patient is to be transferred; providing a patient transfer information creation interface for generating patient transfer information to be sent to the determined recommended referral hospital; acquiring patient transfer input information through the provided patient transfer information creation interface; generating a patient transfer portfolio on the basis of the acquired patient transfer input information; and sending the generated patient transfer portfolio to the recommended referral hospital.

### BACKGROUND

### Field

The present disclosure relates to a device and a method for providing a patient transfer mediation service.

More specifically, the present disclosure relates to a device and a method for mediating transfer of a patient from any one hospital to another specific hospital.

### Related Art

In general, the exchange of information of medical treatment is necessary in various situations such as when a patient is transferred for medical treatment from primary or secondary medical institutions such as a lower medical institution, private hospitals or small and medium-sized hospitals to an upper medical institution, university hospitals, general hospitals, etc., or when a patient medical treatment request is performed between medical institutions in different medical fields.

For example, the exchange of information of medical treatment is necessary when a patient visiting a private hospital is transferred for medical treatment to a general hospital or when a patient is transferred for medical treatment to a medical institution in another medical field of the same level.

Conventionally, the exchange of medical treatment information is performed in the form of a patient taking a memo of medical treatment information received from a lower medical institution and then bringing the memo to an upper medical institution to present the same.

Recently, with the spread of the Internet, there has been an attempt to exchange medical treatment information directly between lower medical institutions and upper medical institutions online instead of having patients bring medical treatment information.

For example, as a system that provides current medical treatment information of a patient online, there is a method using the homepage of a university hospital or general hospital in the most common form.

In detail, the exchange of medical treatment information is performed in the form such that a private hospital accesses the homepage of a hospital for sending a patient from the private hospital, fills out a medical treatment request form in accordance with the form required by the corresponding hospital, and attaches related materials to provide medical treatment information, or performed in the form of a hospital/clinic (doctor) or patient requesting medical treatment through access to inquire medical treatment information.

However, these systems are vulnerable to a security breach and is merely a concept of information inquiry, not information exchange, so detailed delivery of information is impossible, and only inquiry by access is possible.

In addition, it is very difficult to efficiently exchange medical treatment information when all types of equipment used are different between hospitals, and there are issues such as requiring separate equipment to enable the same.

In addition, although doctors who recommend hospitals do not have any obligation to exchange information, they have the inconvenience of accessing the systems of other hospitals and filling out medical treatment information in accordance with the format provided by the systems provided by each different hospital. Hence, most of them merely recommend hospitals, but there are almost no doctors who even exchange information.

Meanwhile, with the development of information technology (IT), various technologies and systems for providing customized medical services specialized to each patient are being developed and introduced.

For example, these digital systems record the condition or medical treatment process of a patient as objective data such as an Electronic Medical Record (EMR) or Electronic Health Record (EHR), and enable utilization and data processing in a wide range of fields based on the recorded data.

Related Patent Document 1 discloses a system capable of sharing medical treatment information between a private hospital and a large hospital using IT technology without violating the current medical law. However, the technology of Related Patent Document 1 focuses on receiving medical treatment information or EMR data from the perspective of a large hospital, and thus hospitals sending patients have the disadvantage of having to do manual work such as organizing materials to send medical treatment information or EMR data to large hospitals.

In detail, Related Patent Document 1 does not provide any convenience for the information sending side, and thus poses an issue that doctors who are still reluctant or bothered to exchange information are less likely to use the system.

In addition, current information exchange is focused on the exchange of an EMR, and there is an issue in that it is difficult to include consultation details not recorded in the EMR in information exchange.

### SUMMARY

An aspect of the present disclosure is to provide a device and a method for providing a patient transfer mediation service capable of securing continuity and convenience of medical treatment by electronically sending the medical treatment history of a patient using computer technology and network technology, rather than directly transferring patient treatment information in the form of a medium such as CD or an offline paper chart printout by the patient when the patient is transferred for medical treatment from any one hospital to another specific hospital.

In addition, an aspect of the present disclosure is to provide a patient transfer mediation service that integrates the patient medical treatment request procedures for each hospital into one system from the perspective of a recommender, in order to solve the inconvenience of requesting patient medical treatment by performing different patient medical treatment request procedures according to each hospital system.

In addition, an aspect of the present disclosure is to provide a patient transfer mediation service that unifies and provides patient medical treatment information having various forms in one format.

In addition, an aspect of the present disclosure is to provide a patient transfer mediation service utilizing a digital system that provides customized medical services for each patient based on information technology (IT).

In addition, an aspect of the present disclosure is to provide a patient transfer mediation service capable of easily creating patient medical treatment information, easily editing numerous materials of a transfer-requesting hospital at the transfer-requesting hospital, and sending only necessary information to a referral hospital.

Technical aspects to be achieved by the present disclosure and embodiments according to the present disclosure are not limited to the technical aspects described above, and other technical aspects may also be addressed.

A device and a method for providing a patient transfer mediation service according to an embodiment of the present disclosure correspond to a patient transfer mediation service providing method of transferring a patient to a referral hospital by a terminal of a transfer-requesting hospital, wherein the method includes: outputting a provisionally-recommended referral hospital list including at least one provisionally-recommended referral hospital to which the patient is to be transferred; determining, from the output provisionally-recommended referral hospital list, a recommended referral hospital to which the patient is to be transferred; providing a patient transfer information creation interface for generating patient transfer information to be sent to the determined recommended referral hospital; acquiring patient transfer input information through the provided patient transfer information creation interface; generating a patient transfer portfolio on the basis of the acquired patient transfer input information; and sending the generated patient transfer portfolio to the recommended referral hospital.

In this connection, the outputting of the provisionally-recommended referral hospital list includes: setting a condition for the referral hospital; and detecting the at least one provisionally-recommended referral hospital matching the condition of the set referral hospital, wherein the condition of the referral hospital includes at least one of a size of the referral hospital, a department of medical treatment, and a distance from the transfer-requesting hospital.

In addition, the providing of the patient transfer information creation interface includes providing a patient transfer information creation template including an input window for inputting information of the transfer-requesting hospital and a setting window for setting whether to include the information in patient transfer information among a plurality of pieces of patient medical treatment information.

In addition, the generating of the patient transfer portfolio on the basis of the acquired patient transfer input information includes: extracting the patient medical treatment information based on the patient transfer input information; and integrating the extracted patient medical treatment information and generating the same as a single file.

In addition, the generating of the patient transfer portfolio on the basis of the acquired patient transfer input information further includes performing automatic reduction editing to reduce a size of the patient transfer portfolio.

The method further includes generating a processed version of the patient transfer portfolio by providing a modification interface for a user to modify the output patient transfer portfolio.

In addition, the device and the method for providing the patient transfer mediation service according to an embodiment of the present disclosure further includes acquiring consent from the patient and medical personnel to send the patient transfer portfolio to the recommended referral hospital, wherein the patient medical treatment information includes at least one of electronic medical records, electronic health records, and medical consultation content.

The device and the method for providing the patient transfer mediation service according to an embodiment of the present disclosure are configured to send patient medical treatment information, which is the medical treatment history information of a patient, from any hospital to another specific hospital in an electronic transfer method using Information and Communications Technologies (ICT), thereby effectively exchanging data necessary for patient medical treatment request, and increasing the continuity and convenience of patient medical treatment between different hospitals.

In addition, the device and the method for providing the patient transfer mediation service according to an embodiment of the present disclosure share patient medical treatment information in an electronic transfer method when a patient requests medical treatment, thereby increasing medical collaboration power according to the formation of a hot line, and creating effects such as shortening medical treatment time and expanding the scope of information access.

In addition, the device and the method for providing the patient transfer mediation service according to an embodiment of the present disclosure share patient medical treatment information in an electronic transfer method when a patient requests medical treatment, so that a patient can be quickly and conveniently transferred from any hospital to another hospital, inconvenience for preparing patient medical treatment information can be reduced, and effects such as reduction of double prescriptions and reduction of medical expenses can be provided.

Moreover, the device and the method for providing the patient transfer mediation service according to an embodiment of the present disclosure can provide an efficient patient transfer mediation service through a consistent interface by integrating and providing patient medical treatment request procedures for each hospital into one system.

In addition, the device and the method for providing the patient transfer mediation service according to an embodiment of the present disclosure integrates and provides various forms of patient medical treatment information as a single file, so that patient medical treatment information can be more conveniently checked and utilized.

In addition, the device and the method for providing the patient transfer mediation service according to an embodiment of the present disclosure provides a patient transfer mediation service using a digital system that provides customized medical services for each patient, thereby more effectively designing medical consultation and treatment plans in the course of patient medical treatment, generating objective data-based feedback based thereon, and performing patient medical treatment using advanced technology.

Moreover, the device and the method for providing the patient transfer mediation service according to an embodiment of the present disclosure can provide a patient transfer mediation service within a range that does not violate the medical law by proceeding with an authentication procedure for approval of data transmission and reception during data exchange between hospital terminals or between hospital terminals and a patient transfer mediation server.

In addition, the device and the method for providing the patient transfer mediation service according to an embodiment of the present disclosure provides a predetermined reward to a hospital that requested a patient when the medical treatment of the corresponding patient is completed through the patient transfer mediation service, so that hospitals can be motivated to use this service more actively to perform patient medical treatment requests, and reasonable compensation can be provided to implement a patient transfer mediation service that is operated reasonably.

The benefits of the present disclosure are not limited to those mentioned above, and other benefits not mentioned herein will be clearly understood by those skilled in the art from the following description.

### BRIEF DESCRIPTION OF THE DRAWINGS

FIG. 1 is a conceptual diagram of a system for providing a patient transfer medication service according to an embodiment of the present disclosure.

FIG. 2 is an internal block diagram of a terminal according to an embodiment of the present disclosure.

FIG. 3 shows an example of an external appearance of a terminal according to an embodiment of the present disclosure.

FIG. 4 is an internal block diagram of a patient transfer mediation server according to an embodiment of the present disclosure.

FIG. 5 is a diagram for explaining some functions of a patient transfer mediation server according to an embodiment of the present disclosure.

FIG. 6 is a flowchart illustrating a method for providing a patient transfer mediation service according to an embodiment of the present disclosure.

FIG. 7 is an example of providing a consultation input interface on medical consultation assistant content output through a terminal according to an embodiment of the present disclosure.

FIG. 8 is an example of a patient transfer information creation template according to an embodiment of the present disclosure.

FIGS. 9 to 11 are diagrams for explaining some operations in a process of generating a patient transfer portfolio according to an embodiment of the present disclosure.

FIG. 12 is a diagram for explaining a patient transfer portfolio processed version according to an embodiment of the present disclosure.

FIG. 13 is an example of receiving signatures of a patient and medical personnel according to an embodiment of the present disclosure.

### DESCRIPTION OF EXEMPLARY EMBODIMENTS

Embodiments of the present disclosure will be shown in the drawings and described in detail through a detailed description. However, the present disclosure may be variously modified and embodied in a variety of different embodiments. Advantages and features of the present disclosure and implementation methods thereof will be clarified through the following embodiments described with reference to the accompanying drawings. Therefore, the present disclosure is not limited to these embodiments introduced hereinafter and might be embodied in a different shape from these embodiments. The terms “first,” “second,” and so on in the present disclosure are used for distinguishing one component from the other components, but they do not specify limited meanings. Also, the singular forms used in the present disclosure are intended to include the plural forms, unless the context clearly indicates otherwise. Moreover, the terms “comprises” and/or “having” described in the present disclosure specify the presence of stated components and/or features, but do not preclude the presence or addition of one or more other components and/or features. Furthermore, the size or the thickness of each component in the drawings can be exaggerated or reduced for the definiteness of explanation. For example, the size and the thickness of each component in the drawings are arbitrarily represented for the convenience of explanation. In accordance therewith, the present disclosure is not limited to the matters shown in the drawings.

Reference will now be made in detail to the embodiments of the present disclosure with reference to the accompanying drawings. Wherever possible, the same reference numbers will be used throughout the disclosure including the drawings to refer to the same or like parts. As such, the repeatable description of the same or like parts will be omitted.

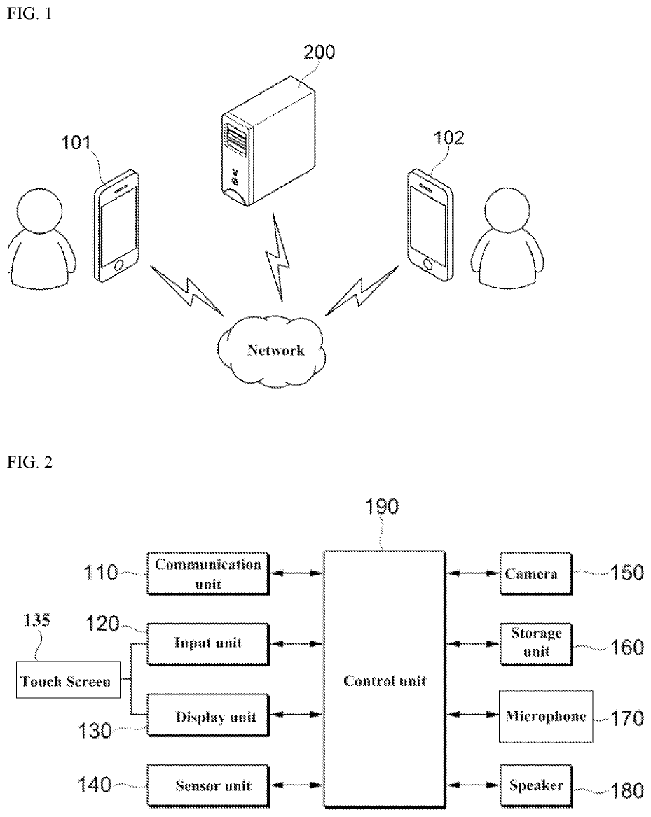

FIG. 1 is a conceptual diagram of a system for providing a patient transfer medication service according to an embodiment of the present disclosure.

Referring to FIG. 1, the system for providing the patient transfer medication service may include a terminal 100, and a patient transfer mediation server 200.

Herein, each component of FIG. 1 may be connected through a network.

In detail, the network refers to a connection structure capable of exchanging information between nodes such as the terminal 100 and the patient transfer mediation server 200. Examples of the network include 3GPP (3rd Generation Partnership Project) network, LTE (Long Term Evolution) network, WIMAX (World Interoperability for Microwave Access) network, Internet, LAN (Local Area Network), Wireless LAN (Wireless Local Area Network), WAN (Wide Area Network), PAN (Personal Area Network), Bluetooth network, Satellite Broadcasting Network, Analog Broadcasting Network, and DMB (Digital Multimedia Broadcasting) network, but are not limited thereto.

Hereinafter, for effective description according to an embodiment of the present disclosure, hospitals will be described by being limited to hospitals registered in the patient transfer mediation system.

Terminal

First, in an embodiment of the present disclosure, the terminal 100 may include a smart phone, a digital broadcasting terminal 100, a mobile phone, a PDA (personal digital assistant), a PMP (portable multimedia player), a navigation device, a tablet PC, a wearable device, and smart glasses, which are portable terminals in which a program for performing a patient transfer mediation service is installed.

In addition, the terminal 100 may further include devices in which a program for executing a method of assisting medical consultation based on wired/wireless communication, such as personal computers is installed, which include a desktop PC, which is a fixed terminal, a laptop computer, and an ultrabook.

The role of the terminal 100 may be divided according to the subject used in an embodiment of the present disclosure.

In detail, in an embodiment, the terminal 100 may be divided into a transfer-requesting hospital 101 used by a transfer-requesting hospital and a referral hospital terminal 102 used by a referral hospital.

First, the transfer-requesting hospital refers to the terminal 100 used by the transfer-requesting hospital, which is a hospital that intends to transfer a patient to another hospital (for example, referral hospital), and may further include a server of the transfer-requesting hospital.

The transfer-requesting hospital terminal 101 may provide a patient transfer mediation service that drives a patient transfer mediation program and performs a patient transfer request to a referral hospital and transfer of related information.

In detail, the transfer-requesting hospital terminal 101 may generate transfer information to be provided to a referral hospital when a patient is transferred based on patient medical treatment information, which is information about medical treating a patient who wants to request medical treatment, and may send the generated transfer information to any referral hospital terminal 102 to remotely perform patient medical treatment request.

The referral hospital terminal 102 refers to the terminal 100 used in the referral hospital, which is a hospital capable of taking over patients from a transfer-requesting hospital, and may further include a server of the referral hospital.

The referral hospital terminal 102 may receive and provide transfer information about a patient who has received a request for medical treatment from the transfer-requesting hospital terminal 101 together with a request for medical treatment.

The roles of the transfer-requesting hospital 101 and the referral hospital terminal 102 as described above are divided according to the subject of use, and functional characteristics of the terminal 100 as described below may be the same.

Hereinafter, each component configuring the terminal 100 will be described in detail with reference to the accompanying drawings.

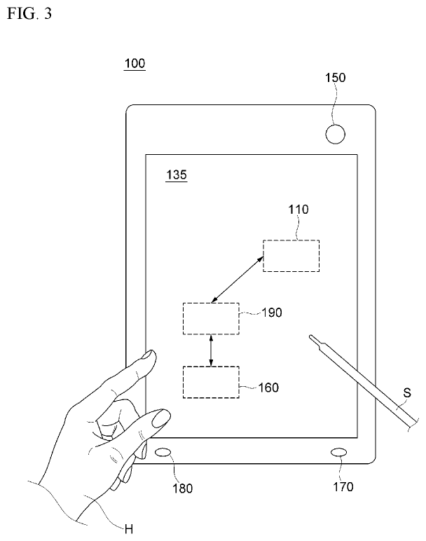

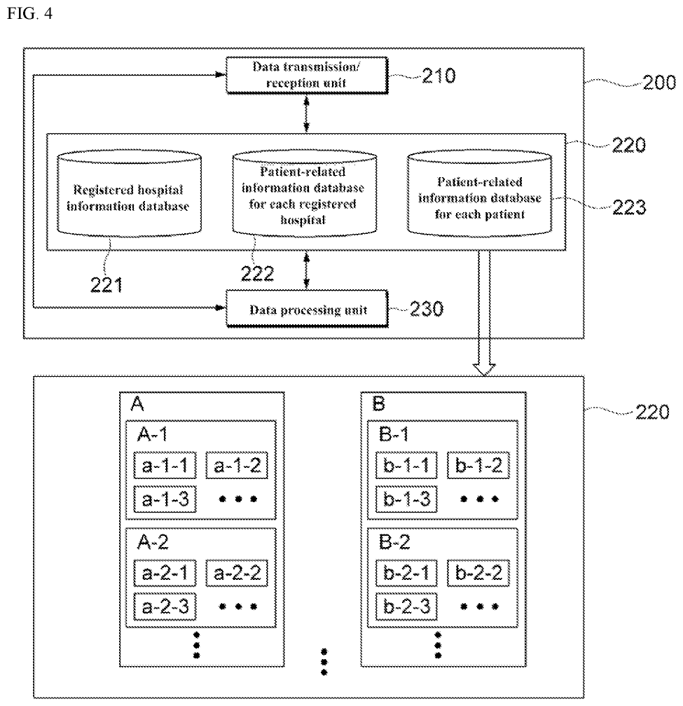

FIG. 2 is an internal block diagram of the terminal 100 according to an embodiment of the present disclosure. FIG. 3 shows an example of an external appearance of the terminal 100 according to an embodiment of the present disclosure.

Referring to FIGS. 2 and 3, the terminal 100 may include a communication unit 110, an input unit 120, a display unit 130, a touch screen 135, a sensor unit 140, a camera 150, a storage unit 160, a microphone 170, a speaker 180, and a control unit 190.

First, the communication unit 110 may transmit and receive various types of data and/or information to provide the patient transfer mediation service.

In an embodiment, the communication unit 110 may communicate with the terminal 100 of another user and/or the patient transfer mediation server 200 to transmit/receive data related to the patient transfer service (for example, patient medical treatment information).

In addition, the communication unit 110 may transmit the patient medical treatment information to an external printer and assist in printing the same as offline printout.

The communication unit 120 may transmit/receive wireless signals to/from at least one of a base station, the external terminal 100, and a certain server on a mobile communication network constructed in accordance with technical standards or communication methods for mobile communication (for example, GSM (Global System for Mobile communication), CDMA (Code Division Multi Access), HSDPA (High Speed Downlink Packet Access), HSUPA (High Speed Uplink Packet Access), LTE (Long Term Evolution), and LTE-A (Long Term Evolution-Advanced)).

Next, the input unit 120 may detect a user input related to the patient transfer mediation service.

In an embodiment, the input unit 120 may detect a user input for creating transfer information, which is information related to the transfer of a patient requesting medical treatment to be sent from a transfer-requesting hospital to a referral hospital, receive a signature of a patient and/or medical personnel, and detect the user input through the consultation input interface provided in the course of medical consultation.

Next, the display unit 130 may output various items of information related to the patient transfer mediation service as a graphic image.

The display unit 130 may include at least one of a liquid crystal display (LCD), a thin film transistor liquid crystal display (TFT LCD), an organic light emitting diode (OLED), a flexible display, a 3D display, and an e-ink display.

In addition, the input unit 120 and the display unit 130 may be combined to be implemented as the touch screen 135.

Next, the camera 150 may capture medical treatment and consultation processes to acquire an image of the medical consultation content, and the terminal 100 that has acquired the image may include the acquired image in the patient medical treatment information.

In addition, the camera 150 may be disposed on the front or/or rear side of the terminal 100 to acquire an image by capturing the disposed direction side, and may be disposed outside the terminal 100 to capture the medical treatment process from an external viewpoint.

When the camera 150 is disposed outside the terminal 100, the camera 150 may send an image of the captured medical treatment process to the control unit 190 through the communication unit 110.

In addition, the camera 150 may include an image sensor and an image processing module.

In detail, the camera 170 may process still images or videos which are acquired by an image sensor (for example, CMOS or CCD).

In addition, the camera 150 may process a still image or video acquired through an image sensor using an image processing module to extract necessary information and transfer the extracted information to the control unit 190.

Next, the storage unit 160 may store any one or more of various application programs, data, and commands for providing the patient transfer mediation service according to an embodiment of the present disclosure.

In an embodiment, the storage unit 160 may store and manage various types of patient medical treatment information and/or transfer information.

The storage unit 160 may be various storage devices such as an ROM, an RAM, EPROM, a flash drive, and a hard drive, and may be a web storage that performs the storage function of the memory 160 on the Internet.

Next, the sensor unit 140 may sense fingerprint information of a patient and/or a counselor, and may assist in proceeding a signature consent procedure based on the sensed fingerprint information.

Next, the microphone 170 may detect a voice input of a patient and/or a counselor during a medical consultation process, and may acquire consultation content recording information based on the detected voice.

In this connection, the terminal 100 that has acquired the consultation content recording information may include the acquired recording information in the patient medical treatment information.

Next, the speaker 180 may output audio information related to the patient transfer mediation service.

In an embodiment, the speaker 180 may output and provide the consultation content recording information included in patient medical treatment information.

Finally, the control unit 190 may control the overall operation of each of the components described above in order to provide the patient transfer mediation service.

The control unit 190 may be implemented using at least one of application specific integrated circuits (ASICs), digital signal processors (DSPs), digital signal processing devices (DSPDs), programmable logic devices (PLDs), field programmable gate arrays (FPGAs), controllers, micro-controllers, microprocessors, and electronic units for performing other functions.

However, the components illustrated in FIGS. 2 and 3 are not essential to implement the terminal 100, so the terminal 100 described in this specification includes more or fewer components than the components listed above.

Patient Transfer Mediation Server

Next, in an embodiment of the present disclosure, the patient transfer mediation server 200 may register and manage hospitals using the patient transfer mediation service in the patient transfer mediation system.

In addition, the patient transfer mediation server 200 may receive various pieces of information about a patient to be transferred to a referral hospital from the transfer-requesting hospital terminal 101, and generate and provide transfer information to be sent to the referral hospital upon request for patient transfer.

Furthermore, the patient transfer mediation server 200 may store and manage the received various pieces of information into a database.

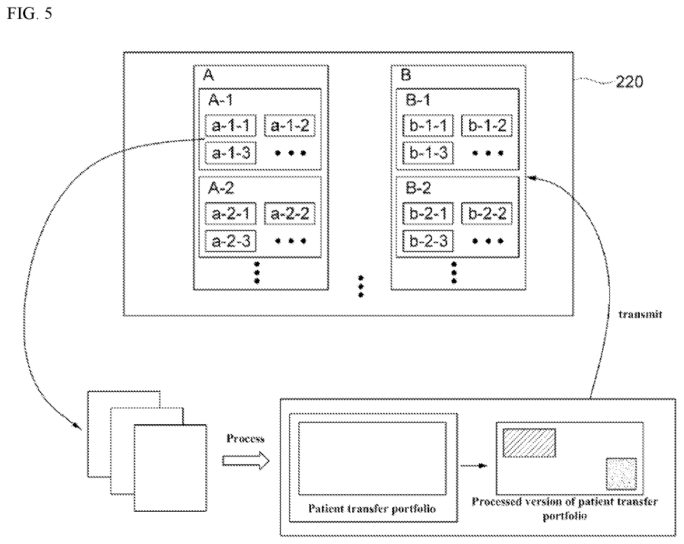

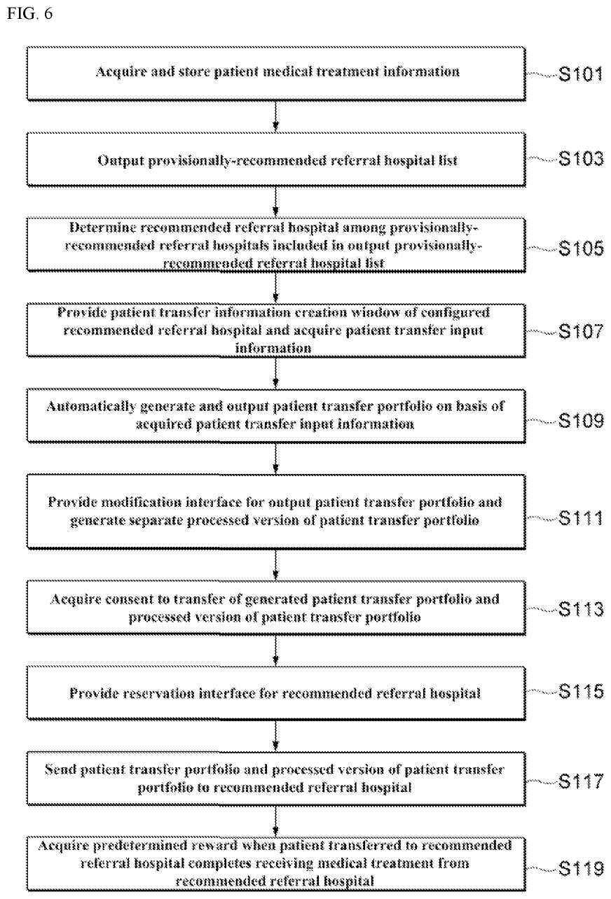

In detail, referring to FIGS. 4 and 5, the patient transfer mediation server 200 may include a data transmission/reception unit 210, a database 220 and a data processing unit 230.

First, the data transmission/reception unit 210 may transmit/receive data for providing the patient transfer brokerage service with a terminal and/or an external server through a network.

Next, the database 220 may store various pieces of data related to the patient transfer mediation service.

In detail, the database 220 may include a registered hospital information database 221, a patient-related information database 222 for each registered hospital, and a patient-related information database 223 for each patient.

More specifically, first, the registered hospital information database 221 may store information related to registered hospitals registered in the patient transfer mediation system.

For example, the registered hospital information database 221 may store hospital names, hospital locations, hospital business registration numbers, hospital phone numbers and/or hospital reservation status information of the hospitals registered in the system (for example, a first hospital A, a second hospital B, . . . ).

In addition, the registered hospital information database 221 may be interlocked with, or further include, at least one of the patient-related information for each registered hospital and the patient-related information database 223 for each patient. In other words, the registered hospital information database 221 may further store and manage information stored in at least one of patient-related information for each registered hospital and patient-related information database 223 for each patient.

Next, the patient-related information database 222 for each registered hospital may classify and store patient-related information for each hospital registered in the system.

In detail, the patient-related information database 222 for each registered hospital may match, store, and manage information related to each hospital and a patient who has a history of medical treatment at each hospital.

For example, the patient-related information database 222 for each registered hospital may store information related to registered patients for each hospital (for example, a first patient A-1, a second patient A-2, . . . , of the first hospital A, or a first patient B-1, a second patient B-2, . . . of the second hospital B) and/or patient classification standard information.

In addition, the patient-related information database 222 for each registered hospital may be interlocked with, or further include the patient-related information database 223 for each patient. In other words, the patient-related information database 222 for each registered hospital may further store and manage information stored in the patient-related information database 223 for each patient.

Next, the patient-related information database 223 for each patient may store and manage patient medical treatment information for each patient matched with each hospital and stored therein.

For example, the patient-related information database 223 for each patient may store at least one of an electronic medical record a-1-1, an electronic health record a-1-2, and medical consultation content a-1-3 of the first patient A-1 of the first hospital A, and may store at least one of an electronic medical record a-2-1, an electronic health record a-2-2, and medical consultation content a-2-3 of the second patient A-1.

In addition, also as for the second hospital B, the patient-related information database 223 for each patient may store at least one of an electronic medical record b-1-1, an electronic health record b-1-2, and medical consultation content b-1-3 of the first patient B-1 of the second hospital B, and may store at least one of an electronic medical record b-2-1, an electronic health record b-2-2, and medical consultation content b-2-3 of the second patient B-1.

The database 140 may be various storage devices such as a ROM, a RAM, an EPROM, a flash drive, and a hard drive, and may be a web storage that performs the storage function of the database 140 on the Internet.

In other words, in an embodiment of the present disclosure, the patient transfer mediation server 200 including such the database 220 may extract and use various pieces of data necessary to provide the patient transfer mediation service from the specific database 220 in which the corresponding data is stored.

In an embodiment, the patient transfer mediation server 200 may extract necessary patient medical treatment information from the patient-related information database 223 for each patient, and generate a patient transfer portfolio based on the extracted information to provide the same to the transfer-requesting hospital terminal 101.

In addition, the transfer-requesting hospital terminal 101 receiving the patient transfer portfolio from the patient transfer mediation server 200 may generate a processed version of the patient transfer portfolio by processing the provided patient transfer portfolio according to an input of a user (for example, medical personnel) or/and according to a template for each referral hospital provided by the referral hospital, and may send the generated patient transfer portfolio processed version and the provided patient transfer portfolio to the specific referral hospital terminal 102.

Subsequently, the referral hospital terminal 102 that has received the patient transfer portfolio and the processed version of the patient transfer portfolio may store and manage the received information in the database 220.

In addition, a more detailed description will be described in the detailed description of a method for providing a patient transfer mediation service described below.

The data processing unit 230 may control the overall operation of each of the components described above in order to provide the patient transfer mediation service.

The data processing unit 230 may be implemented using at least one of the ASICs, DSPs, DSPDs, PLDs, FPGAs, controllers, micro-controllers, microprocessors, and electronic units for performing other functions.

Method for Providing Patient Transfer Mediation Service

Hereinafter, a method for providing a patient transfer mediation service through the patient transfer mediation system will be described in detail mainly based on the transfer-requesting hospital terminal 101.

First, the patient transfer mediation program for providing the patient transfer mediation service may be provided through a network, and may be installed in the storage unit 160 of the transfer-requesting hospital terminal 101 and may be read and driven by the controller 190 through the RAM.

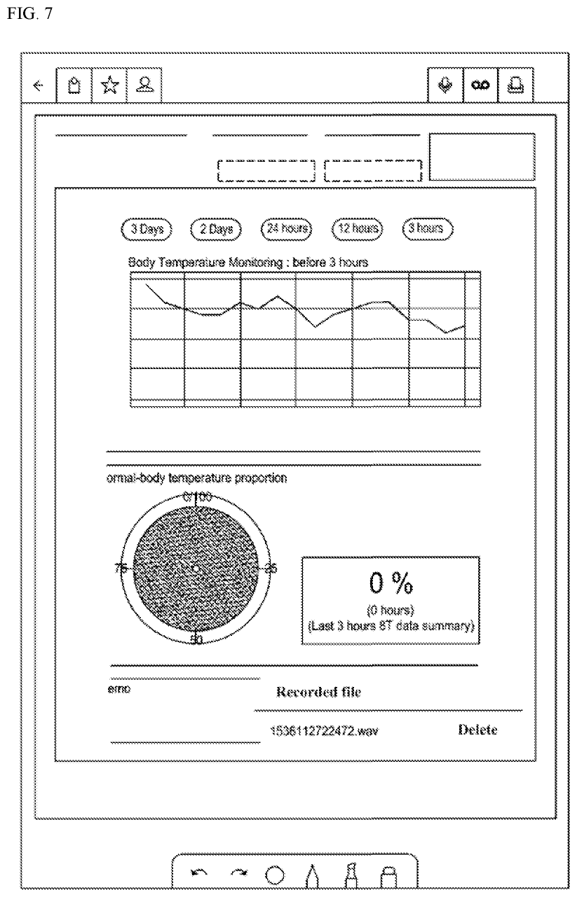

FIG. 6 is a flowchart illustrating a method for providing a patient transfer mediation service according to an embodiment of the present disclosure.

Referring to FIG. 6, the transfer-requesting hospital terminal 101 (hereinafter referred to as hospital terminal) may acquire and store patient medical treatment information (S101).

In detail, the hospital terminal 101 may acquire and store patient medical treatment information including at least one of electronic medical records, electronic health records, and medical consultation content.

Herein, the Electronic Medical Record (EMR) is a computerized patient chart written on existing paper, and may be data information including at least one of personal data of a patient, past medical history, diagnosis record, treatment details, medication guidance record, medication intake record, surgery record, hospitalization and discharge records, and outpatient medical treatment situation information.

The electronic medical record may be stored in the terminal 100 for each hospital because security of information is important for individual privacy protection.

In addition, the Electronic Health Record (EHR) is medical-related data in digital format measured by a device that acquires data, and includes biometric information acquired from a device worn by a patient.

For example, the biometric information may include information obtained by measuring the body of a patient, such as blood sugar, blood pressure, body temperature, and electrocardiogram, or input information about the patient's medication or pain, such as insulin injection information, asthma inhaler injection information, and pain information.

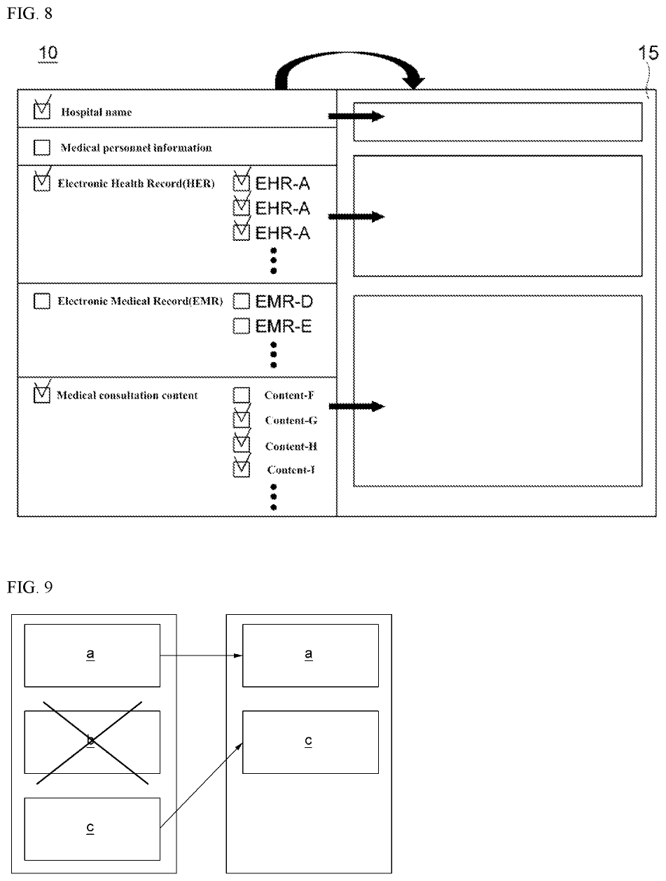

Referring to FIG. 7, the medical consultation content may be information generated by adding a record related to medical consultation input by medical personnel (for example, a doctor) to the medical consultation assistant content.

In this connection, the medical consultation assistant content is information that may assist medical consultation by being formed in a combination including at least one of medical images, videos, animations, and texts and output through the terminal 100, and may include an image assisting consultation provided by the patient transfer mediation server 200, EHR, or EMR.

In detail, the assistant medical consultation content provided by the patient transfer mediation server 200 may include audio-visual materials to assist medical consultation. In an embodiment, the audio-visual materials may include disease diagnosis content, treatment method content, medication consultation content, medical cost content, insurance information content, signature content, other content, and medical consultation assistant content that integrates the aforementioned content.

For example, the medical consultation assistant content may include at least one image or at least one piece of information of a body organ image, disease information, treatment method image, treatment method information, drug taking method, drug information, and insurance information, and may also include various types of information used to create medical consultation content as an assistance.

In addition, in an embodiment, the medical consultation assistant content may be visual content generated based on patient medical treatment information.

Such various pieces of medical consultation assistant content may be displayed on the terminal 100 to be utilized for medical consultation according to the selection of a doctor, and the terminal 100 may arrange and provide a plurality of pieces of selected medical consultation assistant content so that the doctor may easily utilize the same for consultation.

Further, in order to generate medical consultation content by receiving medical consultation details of medical personnel on the medical consultation assistant content, the terminal 100 may provide a consultation input interface.

In detail, the consultation input interface may include a graphic user interface that enables medical personnel input writing, voice, editing, and/or images on the displayed image of medical consultation assistant content.

For example, the consultation input interface may provide a writing input interface that detects writing input of a doctor on the image of the medial consultation assistant content.

In other words, the medical consultation content may be information generated by adding the medical consultation details input through the consultation input interface to the aforementioned medical consultation assistant content, and is output through the terminal 100 to play a role in assisting medical consultation.

As such, the patient transfer mediation service according to an embodiment of the present disclosure utilizes medical consultation content that may provide customized medical services for each patient, thereby more effectively designing medical consultation and treatment plans in the course of patient medical treatment, generating objective data-based feedback based thereon, and performing patient medical treatment using advanced technology.

Returning to the description of the patient transfer mediation service, the hospital terminal 101 may generate the aforementioned patient medical treatment information or acquire the same by receiving the same from the patient transfer mediation server 200.

In addition, the hospital terminal 101 may remotely receive and acquire medical information (for example, self-measurement information of insulin level, self-measurement information of blood pressure level, etc.) measured by a patient through an arbitrary measuring device.

In addition, the hospital terminal 101 may provide an interface through which patient medical treatment information may be input, so that the patient medical treatment information may be acquired by input of medical personnel who has performed medical consultation with a patient.

As such, in the terminal 100 of a hospital using the patient transfer mediation program in an embodiment of the present disclosure, various types of patient medical treatment information are accumulated, and then extracted from the storage unit 160 when necessary to be utilized for medical treatment.

Subsequently, when attempting to transfer a patient having such patient medical treatment information to a referral hospital, the hospital terminal 101 may output a list of provisionally-recommended referral hospitals through a patient transfer mediation program (S103).

In detail, the hospital terminal 101 may operate the patient transfer mediation program in response to a request to use the patient transfer mediation service, and may provide a list of provisionally-recommended referral hospitals.

In this connection, first, the hospital terminal 101 may provide a referral hospital condition input window through which conditions for the hospital to which a patient is to be transferred may be input.

In detail, the hospital terminal 101 may receive condition information including at least one of a size of the referral hospital, a department of medical treatment, and a distance from a transfer-requesting hospital required through the referral hospital condition input window.

In addition, the hospital terminal 101 may search for and derive at least one hospital that meets the condition information acquired through the referral hospital condition input window among the hospitals registered in the patient transfer mediation system, and may set at least one derived hospital as a provisionally-recommended referral hospital which is a candidate referral hospital.

Subsequently, the hospital terminal 101 may arrange the set provisionally-recommended referral hospitals according to a predetermined criterion (for example, order of conformity with condition information, alphabetical order of hospital names, etc.) and output the same as a list of provisionally-recommended referral hospitals.

In this connection, the hospital terminal 101 may include and provide information on any one or more of the hospital name, hospital location, and reservation status of the provisionally-recommended referral hospitals in the list of the provisionally-recommended referral hospitals.

The hospitals registered in the patient transfer mediation system may perform payment for products that provide a function of being exposed at the top of the list of provisionally-recommended referral hospitals in order to be placed at the top of the list of the provisionally-recommended referral hospitals.

In other words, hospitals may obtain the benefit of increasing the inflow through patient mediation by being placed at the top of the list of provisionally-recommended referral hospitals by paying for products exposed at the top of the list of provisionally-recommended referral hospitals through the patient transfer mediation service.

In addition, hospitals may be provided with the benefits of hospital advertisements through the patient transfer mediation service through payment for a product that exposes advertisements for hospitals together on the list of provisionally-recommended referral hospitals.

Next, the hospital terminal 101 that outputs the list of provisionally-recommended referral hospitals may determine a recommended referral hospital among the provisionally-recommended referral hospitals included in the output list of the provisionally-recommended referral hospitals (S105).

In detail, the hospital terminal 101 may receive an input for selecting a recommended referral hospital, which is a referral hospital to be finally recommended to a patient, from medical personnel who has been provided with a list of provisionally-recommended referral hospitals.

Further, the hospital terminal 101 may determine a recommended referral hospital based on a selection input of a referral hospital recommended by medical personnel.

Next, the hospital terminal 101 may acquire patient transfer input information by providing a creation interface for creating patient transfer information of the determined recommended referral hospital (S107).

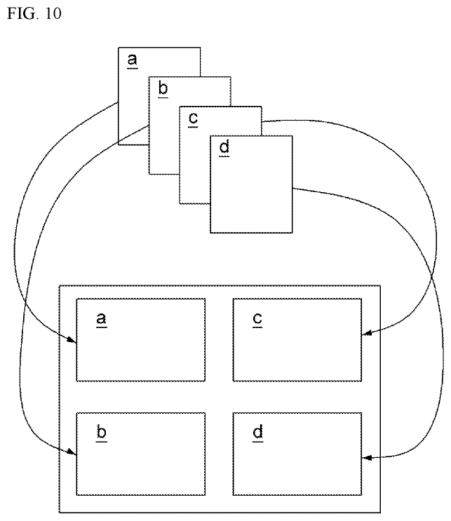

In this connection, referring to FIG. 8, the patient transfer information creation interface may be an interface for generating patient transfer information to be sent to the referral hospital when the transfer-requesting hospital transfers a patient through the patient transfer mediation service.

This patient transfer information creation interface is an interface that sets conditions for generating patient transfer information by editing numerous pieces of patient-related information stored in the transfer-requesting hospital that transfers a patient. Through this patient transfer information creation interface, among numerous pieces of patient-related information, the transfer-requesting hospital may easily generate only the information to be sent as patient transfer information.

The patient transfer information creation interface according to an embodiment may be set to be created according to a template set by a referral hospital to take over a patient.

In detail, when another hospital transfers a patient for medical treatment, a referral hospital presets a patient transfer-requesting template for creating a transfer request including medical treatment information, and later, when a patient is requested to be transferred to the referral hospital, the referral hospital may provide a patient transfer information creation interface based on the preset patient transfer-requesting template.

For example, the referral hospital may set the patient transfer information creation interface, which is divided by hospital, disease, and/or symptom, individually for each category. After generating a template, the referral hospital may induce patient transfer information to be generated according to the set template when a patient is requested to be transferred to the referral hospital.

In other words, the transfer-requesting hospital terminal 101 may provide a patient transfer information creating template 10 to provide a patient transfer information creation interface.

In the embodiment, the patient transfer information creation template 10 may include a window for inputting transfer-requesting hospital information and opinions on a patient, and a window for setting whether to include information on at least one item among electronic health records, electronic medical records, and medical consultation content, which are patient medical treatment information accumulated through the medical treatment of existing patients, in patient transfer information.

In another aspect, the patient transfer information creation template may include: inputting transfer-requesting hospital and patient information; attaching electronic health record (EHR) and electronic medical record (EMR) data; and inserting medical consultation content that records medical consultation details.

In an embodiment, it is possible to input the aforementioned information and create a setting whether to include patient-related information one by one each time a patient is transferred. However, for the convenience of a user, at least portion of the patient transfer information creation template 10 may be provided in a pre-created state.

For example, the patient transfer information creation template 10 may be provided in a state in which information created when a patient is transferred is described.

In detail, in the patient transfer information creation template 10, the transfer-requesting hospital information, such as the name of the transfer-requesting hospital and the name of the medical personnel, may be pre-created with the content created when a patient is transferred.

In addition, the patient transfer information creation template 10 may be provided in a state in which the setting for inclusion is made in the same way as the setting for inclusion among transferred patient medical treatment information.

In addition, the patient transfer information creation template 10 may be set based on information requested by the referral hospital to which a patient is to be transferred.

For example, the patient transfer information creation template 10 may be displayed in a state set to include information requested by the referral hospital and then provided for modification by a user.

Thus, the efficiency of medical information exchange can be increased by allowing the transfer-requesting hospital to check and send the information requested by the referral hospital.

When the preset patient transfer information creation template 10 is stored on the patient transfer mediation system by each hospital, the hospital terminal 101 may set a specific hospital as a recommended referral hospital and at the same time automatically provide the same through the hospital terminal 101.

In addition, the hospital terminal 101 may change an input presence/absence setting for each preset item based on the medical personnel input to the preset patient transfer information creation template 10.

For example, the hospital terminal 101 may provide various selection buttons (for example, checkbox type selection buttons, etc.) by matching each item displayed on the patient transfer information creation template 10.

In this connection, the selection button may display a selected or non-selected state based on the input presence/absence setting for each preset item and may be provided.

Further, the hospital terminal 101 may change whether to select a corresponding item based on a medical personnel input to the provided selection button (for example, clicking the selection button).

In addition, the hospital terminal 101 may generate patient transfer input information including information on selected and unselected items through the selection button provided.

Based on such patient transfer input information, patient transfer information may be generated by automatically editing patient-related information at a transfer-requesting hospital.

Thus, the hospital terminal 101 may generate patient transfer input information based on the patient transfer information creation template 10 created by a user.

In this connection, when necessary to transmit and receive data with the patient transfer mediation server 200 in order to comply with the medical law and interlock the patient transfer mediation server 200 with medical information, the hospital terminal 101 may proceed with a procedure for obtaining approval.

In an embodiment, the hospital terminal 101 may proceed with a public certification login procedure through a hospital's unique public certificate in order to be approved for interlocking with the patient transfer mediation server 200.

As such, the hospital terminal 101 may provide an efficient patient transfer mediation service through a consistent interface by providing different patient medical treatment request procedures for each hospital in one integrated method called the patient transfer information creation template 10.

Next, the hospital terminal 101 that has acquired the patient transfer input information may automatically generate and output a patient transfer portfolio based on the acquired patient transfer input information (S109).

In detail, the hospital terminal 101 may send the acquired patient transfer input information to the patient transfer mediation server 200 after performing an approval procedure for data transmission and reception with the patient transfer mediation server 200.

In addition, the patient transfer mediation server 200 that has received patient transfer input information from the hospital terminal 101 may generate a patient transfer portfolio based on the received patient transfer input information.

Herein, the patient transfer portfolio refers to data generated as a single file by integrating various types (for example, txt, gif, pdf, jpeg, avi, mov, way, etc.) of related information about a patient requesting medical treatment to be sent to a recommended referral hospital.

In other words, the patient transfer portfolio is a material in which various types of information are integrated into a single file, and the recommended referral hospital may more easily check information about the patient requesting medical treatment through the patient transfer portfolio.

In more detail, the patient transfer mediation server 200 may extract information to be included in the patient transfer portfolio from the database 220 based on the patient transfer input information.

In addition, the patient transfer mediation server 200 may generate a patient transfer portfolio by integrating various types of information extracted from the database 220 into one.

For example, referring to FIG. 8, the patient transfer mediation server 200 may extract information about EHR-A, EHR-B, and EHR-C (for example, electronic health information acquired through a blood pressure gauge, asthma inhaler, and heart rate monitor) of the electronic health record, and content-G, content-H, and content-I of medical consultation content from the database 220 based on the patient transfer input information.

In addition, the patient transfer mediation server 200 may generate a patient transfer portfolio by integrating the extracted information into a single file.

In addition, the patient transfer mediation server 200 displays a preview screen 15 output to the patient transfer information creation template 10 based on the information extracted from the database 220 based on the patient transfer input information.

In detail, the patient transfer mediation server 200 may send the generated preview screen information to the hospital terminal 101, and the hospital terminal 101 receiving the preview screen 15 information may output and provide the preview screen 15 through the transfer information creation template 10.

In addition, the patient transfer mediation server 200 may receive data necessary for generating a patient transfer portfolio in conjunction with the storage unit 160 of the hospital terminal 101.

In this connection, the patient transfer mediation server 200 may proceed with a procedure for obtaining approval when data transmission/reception with the hospital terminal 101 is necessary in order to comply with the medical law and interlock with the hospital terminal 101.

In an embodiment, the patient transfer mediation server 200 may proceed with a public certification approval procedure through a unique public certificate of the hospital in order to interlock with the hospital terminal 101.

As such, the patient transfer mediation server 200 integrates and provides various forms of patient-related information into a single file, thereby assisting in more conveniently checking and utilizing patient-related information such as patient medical treatment information.

In addition, the patient transfer mediation server 200 may generate a patient transfer portfolio by listing patient-related information extracted from the database 220 or received from the hospital terminal 101 according to a preset arrangement order based on the patient transfer input information.

In detail, the patient transfer mediation server 200 may list patient-related information according to a preset arrangement criterion, such as a preset order of items of high importance and an order individually preset by a transfer-requesting hospital.

For example, the patient transfer mediation server 200 may arrange patient-related information in the order of hospital name, medical personnel information, and patient medical treatment information (in other words, at least one of electronic health records, electronic medical records, and medical consultation content).

In this connection, the patient transfer mediation server 200 may list data for at least one of electronic health records, electronic medical records, and medical consultation content according to a preset arrangement order within the patient medical treatment information.

For example, the patient transfer mediation server 200 may list patient medical treatment information on the patient transfer portfolio according to a preset arrangement criterion, such as a recent information order or a preset order of importance.

In addition, the patient transfer mediation server 200 may list at least one piece of medical consultation content according to a preset order (for example, order of latest information, preset order of importance, etc.) within the medical consultation content with respect to the medical consultation content of the patient medical treatment information.

As such, the patient transfer mediation server 200 arranges and provides patient-related information within the patient transfer portfolio according to preset criteria, so that information can be delivered more effectively and intuitively.

Subsequently, the patient transfer mediation server 200 may execute automatic reduction editing when the patient transfer portfolio to be generated exceeds a predetermined amount and/or includes unnecessary information.

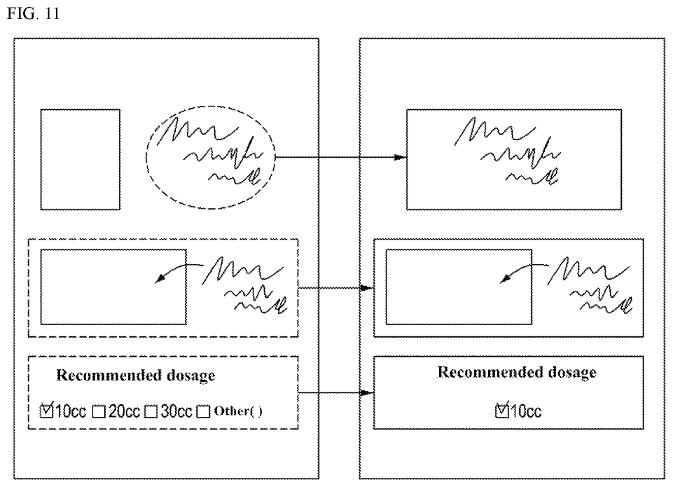

In detail, the patient transfer mediation server 200 may 1) delete patient-related information irrelevant to the characteristics of a department of medical treatment of the recommended referral hospital (FIG. 9).

For example, the patient transfer mediation server 200 may delete unnecessary surgery related medical treatment records included in patient related information when transferring a patient to an internal medical department.

In addition, the patient transfer mediation server 200 may 2) reduce the size of specific information (FIG. 10).

For example, the patient transfer mediation server 200 may reduce the size of the content by classifying at least one piece of medical consultation content by date and re-editing the classified medical consultation content by date into a size of one page.

In addition, the patient transfer mediation server 200 may 3) perform reduction editing by extracting only main information from medical consultation content (FIG. 11).

For example, the patient transfer mediation server 200 may extract only handwritten information recording consultation details by a doctor on medical consultation content.

However, when there is handwritten information recorded in conjunction with information (for example, image information, checkbox information, etc.) other than the handwritten information on the medical consultation content, the patient transfer mediation server 200 may extract all of the corresponding other information and handwritten information.

As such, the patient transfer mediation server 200 executes automatic reduction editing per request of a user when the information acquired to generate the patient transfer portfolio is excessively large or includes unnecessary information, and thus through the patient transfer mediation service, the quality of patient-related information provided to recommended referral hospitals can be improved.

In the above, it has been described that the patient transfer mediation server 200 generates a patient transfer portfolio, but in other embodiments, various embodiments such as performing some operations in the hospital terminal 101 will be possible.

In addition, the patient transfer mediation server 200 that has generated the patient transfer portfolio may send the generated patient transfer portfolio to the hospital terminal 101.

In addition, the hospital terminal 101 that has received the patient transfer portfolio may output the received patient transfer portfolio.

Next, the hospital terminal 101 that has output the patient transfer portfolio may provide a modification interface for the output patient transfer portfolio, and may separately generate a processed version of the patient transfer portfolio (hereinafter, a processed version of the portfolio) (S111).

In detail, the hospital terminal 101 may provide a modification interface including at least one of 1) an interface that provides a function of deleting or blinding privacy information of patients and/or medical personnel, 2) an interface that provides a function of removing records of consultation details (for example, handwriting input, voice input, etc.) at once, 3) an interface that provides a function of extracting only items selected by medical personnel from the patient transfer portfolio, and 4) an interface that extracts, summarizes and provides only preset important items from the patient transfer portfolio.

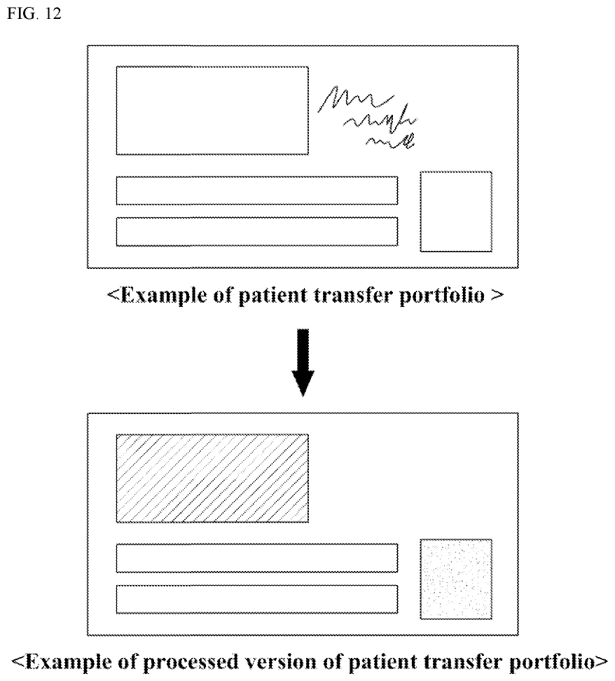

Further, the hospital terminal 101 may generate a processed version of the portfolio as shown in FIG. 12 by reflecting the information input through the modification interface.

As such, the hospital terminal 101 may provide a modification interface that implements interfaces for various purposes, and provide a re-processed version of the patient transfer portfolio based on input through the provided modification interface.

Next, the hospital terminal 101 may acquire the consent of a patient and medical personnel for the transfer of the generated patient transfer portfolio and the processed version of the portfolio (hereinafter, patient portfolio information) (S113).

In detail, the hospital terminal 101 may acquire approval for transfer of patient portfolio information from a patient matched to patient portfolio information to be transferred to the recommending referral hospital and medical personnel in charge of the patient to the recommended referral hospital through signature input.

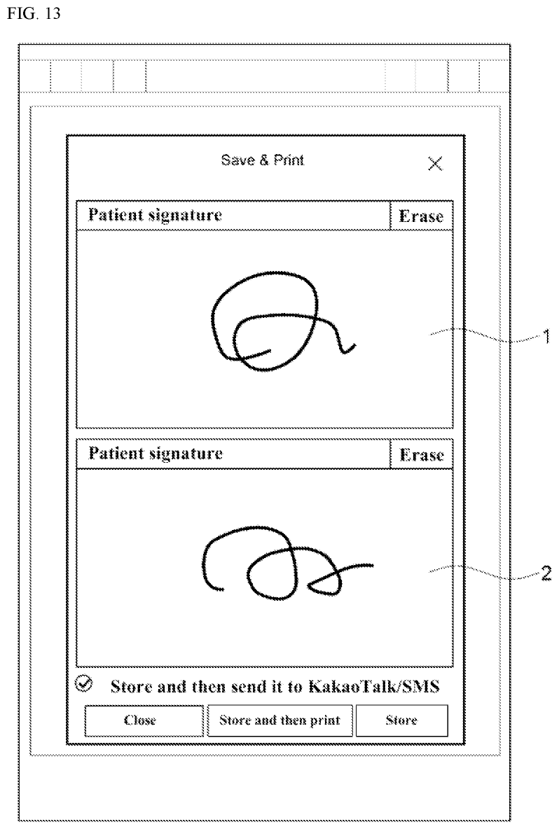

In an embodiment, referring to FIG. 13, the hospital terminal 101 may output a signature input window, receive a signature input of medical personnel through a medical personal signature input window 1, and receive a signal input of a patient through a patient signature input window 2.

In another embodiment, the hospital terminal 101 may receive fingerprint inputs of the patient and medical personnel through the sensor unit 140 to acquire consent for the transfer of patient portfolio information.

As such, the hospital terminal 101 sends data on patient portfolio information after obtaining approval from the patient and medical personnel, thereby realizing information delivery that is lawful under the medical law based on the clear consent of the patient and medical personnel.

In addition, the hospital terminal 101 may provide a reservation interface for the set recommended referral hospital (S115).

In detail, the hospital terminal 101 may output reservation status information of recommended referral hospitals through the reservation interface, and thus, may provide date/time information for medical treatment.

In addition, the hospital terminal 101 may make a medical treatment reservation at a recommended referral hospital based on a medical treatment date/time selection input within a medical treatment available date/time.

As such, the hospital terminal 101 may assist a patient to receive medical treatment at the recommended referral hospital quickly and conveniently by providing a function of reserving medical treatment for the recommended referral hospital.

In addition, the hospital terminal 101 may send the generated patient portfolio information to the recommended referral hospital (S117).

In detail, the hospital terminal 101 may generate patient portfolio information, and after obtaining consent for transfer of the generated patient portfolio information, send the generated patient portfolio information together with a patient transfer request signal to the terminal 10 of the recommended referral hospital.

Herein, the patient transfer request signal may include a patient transfer request alarm, a patient transfer request pop-up window, and the like.

Further, a recommended referral hospital terminal 102 receiving the patient transfer request signal and patient portfolio information may receive a patient transfer acceptance input for approving the transfer of a patient from the transfer-requesting hospital.

In addition, when a patient transfer acceptance input is received, the recommended referral hospital terminal 102 may output and provide the received patient portfolio information.

In an embodiment of the present disclosure, the hospital terminal 101 may also send the generated patient portfolio information to a government agency and/or a private organization such as an insurance company, a pharmaceutical company, a medical device company, a mediation IT company, etc. that requires the corresponding information.

In other words, the hospital terminal 101 may provide the generated patient portfolio information to various government agencies and/or private organizations to assist the patient portfolio information to be utilized in a wide range of fields, and to generate and provide more influential patient-related information.

As such, in an embodiment of the present disclosure, the hospital terminal 101 sends patient portfolio information to the recommended referral hospital terminal 102 so that the patient transfer portfolio or the processed version of portfolio may be all checked as needed, thereby enabling effective viewing of patient-related information at a recommended referral hospital.

In addition, the recommended referral hospital terminal 102 may prevent indiscriminate exposure of patient portfolio information by outputting received patient portfolio information after receiving a patient transfer acceptance input.

In addition, the hospital terminal 101 may acquire a predetermined reward when a patient transferred to a recommended referral hospital completes medical treatment at the recommended referral hospital (S119).

In detail, the hospital terminal 101 may receive a predetermined reward from the patient transfer mediation server 200 through a patient transfer mediation program in various ways when a patient requesting medical treatment at a recommended referral hospital completes medical treatment.

For example, the hospital terminal 101 may acquire a reward in a way of receiving points available in a patient transfer brokerage program.

In this connection, the points available in the patient transfer mediation program may be paid from various organizations such as referral hospitals, government agencies, insurance companies, pharmaceutical companies, medical device companies, and/or mediation IT companies.

As such, the patient transfer mediation service according to an embodiment of the present disclosure provides a predetermined reward to a hospital that requested a patient, so that hospitals can be motivated to use this service more actively to perform patient medical treatment requests, and a reasonable reward can be provided to implement a patient transfer mediation service that is operated reasonably.

The device and the method for providing the patient transfer mediation service according to an embodiment of the present disclosure are configured to send patient medical treatment information, which is the medical treatment history information of a patient, from any hospital to another specific hospital in an electronic transfer method using Information and Communications Technologies (ICT), thereby effectively exchanging data necessary for patient medical treatment request, and increasing the continuity and convenience of patient medical treatment between different hospitals.

In addition, the device and the method for providing the patient transfer mediation service according to an embodiment of the present disclosure share patient medical treatment information in an electronic transfer method when a patient requests medical treatment, thereby increasing medical collaboration power according to the formation of a hot line, and creating effects such as shortening medical treatment time and expanding the scope of information access.

In addition, the device and the method for providing the patient transfer mediation service according to an embodiment of the present disclosure share patient medical treatment information in an electronic transfer method when a patient requests medical treatment, so that a patient can be quickly and conveniently transferred from any hospital to another hospital, inconvenience for preparing patient medical treatment information can be reduced, and effects such as reduction of double prescriptions and reduction of medical expenses can be provided.

Moreover, the device and the method for providing the patient transfer mediation service according to an embodiment of the present disclosure can provide an efficient patient transfer mediation service through a consistent interface by integrating and providing patient medical treatment request procedures for each hospital into one system.

In addition, the device and the method for providing the patient transfer mediation service according to an embodiment of the present disclosure integrates and provides various forms of patient medical treatment information as a single file, so that patient medical treatment information can be more conveniently checked and utilized.

In addition, the device and the method for providing the patient transfer mediation service according to an embodiment of the present disclosure provides a patient transfer mediation service using a digital system that provides customized medical services for each patient, thereby more effectively designing medical consultation and treatment plans in the course of patient medical treatment, generating objective data-based feedback based thereon, and performing patient medical treatment using advanced technology.

Moreover, the device and the method for providing the patient transfer mediation service according to an embodiment of the present disclosure can provide a patient transfer mediation service within a range that does not violate the medical law by proceeding with an authentication procedure for approval of data transmission and reception during data exchange between hospital terminals 100 or between hospital terminals 100 and a patient transfer mediation server.

In addition, the device and the method for providing the patient transfer mediation service according to an embodiment of the present disclosure provides a predetermined reward to a hospital that requested a patient when the medical treatment of the corresponding patient is completed through the patient transfer mediation service, so that hospitals can be motivated to use this service more actively to perform patient medical treatment requests, and a reasonable reward can be provided to implement a patient transfer mediation service that is operated reasonably.

In addition, embodiments of the present disclosure described above may be implemented in the type of program commands the can be executed through various computer components, and may be recorded on a computer-readable recording medium. The computer-readable recording medium may include program commands, data files, and data structures individually or in combinations thereof. The program commands that are recorded on a computer-readable recording medium may be those specifically designed and configured for the present disclosure or may be those available and known to those engaged in computer software in the art. The computer-readable recording medium includes magnetic media such as hard disks, floppy disks, and magnetic media such as a magnetic tape, optical media such as CD-ROMs and DVDs, magneto-optical media such as floptical disks, and hardware devices specifically configured to store and execute program commands, such as ROM, RAM, and flash memory. The program commands include not only machine language codes compiled by a compiler, but also high-level language code that can be executed by a computer using an interpreter etc. A hardware device may be changed into one or more software module to perform the processes according to the present disclosure, and vice versa.

### INDUSTRIAL APPLICABILITY

An embodiment of the present disclosure is directed to a service method for transmitting and receiving patient transfer information between hospitals through a terminal, and thus has industrial applicability.

What is claimed is:

1. A method for providing a patient transfer mediation service of transferring a patient to a referral hospital by a terminal of a transfer-requesting hospital, wherein the method includes:
outputting a provisionally-recommended referral hospital list including at least one provisionally-recommended referral hospital to which the patient is to be transferred;
determining, from the output provisionally-recommended referral hospital list, a recommended referral hospital to which the patient is to be transferred;
providing a patient transfer information creation interface for generating patient transfer information to be sent to the determined recommended referral hospital;
acquiring patient transfer input information through the provided patient transfer information creation interface;
generating a patient transfer portfolio on the basis of the acquired patient transfer input information; and
sending the generated patient transfer portfolio to the recommended referral hospital.

2. The method of claim 1, wherein the outputting of the provisionally-recommended referral hospital list includes:
setting a condition for the referral hospital; and
detecting the at least one provisionally-recommended referral hospital matching the condition of the set referral hospital,
wherein the condition of the referral hospital includes at least one of a size of the referral hospital, a department of medical treatment, and a distance from the transfer-requesting hospital.

3. The method of claim 1, wherein the providing of the patient transfer information creation interface includes providing a patient transfer information creation template including an input window for inputting information of the transfer-requesting hospital and a setting window for setting whether to include the information in patient transfer information among a plurality of pieces of patient medical treatment information.

4. The method of claim 3, wherein the generating of the patient transfer portfolio on the basis of the acquired patient transfer input information includes:
extracting the patient medical treatment information based on the patient transfer input information; and
integrating the extracted patient medical treatment information and generating the same as a single file.

5. The method of claim 4, wherein the generating of the patient transfer portfolio on the basis of the acquired patient transfer input information further includes performing automatic reduction editing to reduce a size of the patient transfer portfolio.

6. The method of claim 5, further including generating a processed version of the patient transfer portfolio by providing a modification interface for a user to modify the output patient transfer portfolio.

7. The method of claim 1, further including acquiring consent from the patient and medical personnel to send the patient transfer portfolio to the recommended referral hospital, wherein the patient medical treatment information includes at least one of electronic medical records, electronic health records, and medical consultation content.
